(19) United States
(12) Patent Application Publication
(10) Pub. No.: US 2017/0215891 A1
(43) Pub. Date: Aug. 3, 2017

(54) A Tourniquet

(71) Applicant: Chase Products Limited, Herefordshire (GB)

(72) Inventor: Gareth JONES, Herefordshire (GB)

(21) Appl. No.: 15/502,176

(22) PCT Filed: Aug. 6, 2015
(86) PCT No.: PCT/GB2015/052278

§ 371 Date: Feb. 6, 2017

(30) Foreign Application Priority Data
Aug. 7, 2014 (GB) 1414003.2

(51) Int. Cl. A61B 17/132

(52) U.S. Cl. CPC A61B 17/1322; A61B 17/1327

(57) ABSTRACT

A tourniquet of flexible material is disclosed. The tourniquet includes an elongate member having a first and a second end, the first end having a notch. The elongate member defines an aperture therethrough, proximate to the first end, for receiving the second end of the elongate member. In use, the elongate member comprises at least one engagement member disposed along its length to engage the aperture.

The present invention relates to a tourniquet and more specifically to a single-use tourniquet.

Tourniquets have been used for years in medical procedures to restrict blood flow or to cause turgidity in veins. They are often used by physicians when taking a blood sample, usually from a patient's arm. The physician places the tourniquet around the patient's arm and tightens the tourniquet to cause turgidity in a vein. The physician can then take a blood sample using venipuncture practices.

Many designs of tourniquets are known. Chinese patent application number CN203153850 describes a tourniquet comprising a flat, elastic band having nylon buckles for maintaining the tourniquet in place. This is a complicated design which involves associated high manufacturing costs. U.S. Pat. No. 5,653,728 describes a disposable tourniquet which is knotted to hold it in place during use. Knotting a tourniquet can be a two-handed process and requires the physician to maintain tension in the tourniquet whilst knotting it which can also require the use of both hands. Other problems associated with the prior art are that tourniquets can become contaminated by blood, hair or bacteria from the patient or the physician. Disposable tourniquets have therefore been suggested.

UK patent application GB2424189 describes a tourniquet formed of a strip of sheet material having apertures through which a latching element can pass and engage. However, the design of this tourniquet results in the sheet material, which has passed through the aperture, becoming curved in use. This reduces its width and results in the latching element becoming disengaged and the tourniquet loosening. The effect can be exacerbated by the patient moving their arm, or body part around which the tourniquet is placed. This can result in the vein no longer being turgid and the physician having to repeat the process. There is therefore a need for a tourniquet which overcomes all of the problems associated with the prior art.

The present invention, in at least some of its embodiments, seeks to solve at least some of the problems mentioned above.

The present invention provides: a tourniquet of flexible material including: an elongate member having a first and a second end, said elongate member defining an aperture there through, proximate to the first end, for receiving the second end of the elongate member when in use so that the elongate member is wrapped around a body part so that the second end can be pulled against the aperture to tighten the elongate member around a limb, said elongate member having at least one engagement member disposed along its length to engage the aperture wherein the first end defines a notch.

The tourniquet can be placed around the patient's limb, usually by a physician, by passing the second end of the elongate member through the aperture so that the tourniquet can be slid over the patient's limb. The second end is then pulled so as to further pass the elongate member through the aperture. The second end is pulled against an end of the aperture closest to the notch so as to exert a force on the patient's limb. The notch allows the first end of elongate member to deform, when in use, so as to ensure that the rest of the elongate member remains flat against the limb of a patient thereby ensuring the tourniquet remains in place. As the first end of the elongate member deforms it causes the aperture to deform as well, thereby exerting a positive engagement on the at least one engagement member which results in the tourniquet remaining in place, even if the patient moves. A further advantage of the design is that it can be used one-handed by a physician. The tourniquet can also be released very easily and even one handed, by deforming the elongate member so that it is dimensioned to pass through the aperture. This can be achieved in a number of ways including pulling on the first end proximate to the notch which causes the aperture to deform the elongate member so that it easily passes through the aperture. The tourniquet can then be slid off the body part once it is sufficiently loosened or the second end can be passed back through the aperture. This quick release minimises the length of time a patient is subjected to pressure around their body part.

The at least one engagement member is configured to engage with the aperture defined by the first end of the elongate member. This can be achieved by any suitable means such as cuts in the elongate body. In an embodiment this is achieved by at least one indentation. In an embodiment, the elongate member has two substantially parallel edges along which the at least one indentation is located.

In an embodiment, the elongate member has a plurality of indentations along its length so that the tourniquet can be used on different sized limbs. In a further embodiment, the indentations are spaced along the length of the elongate body on both of the substantially parallel edges so that there are two points of engagement with the aperture thereby forming a positive engagement.

The indentations may be opposite one another on either side of the elongate member or the indentations can be staggered along the length of the elongate member.

The indentations may have rounded surfaces to avoid tearing of the elongate member when a force is applied to the elongate member.

In an embodiment, the material of construction is a silicone rubber. The silicone rubber is biocompatible and will not cause an allergic reaction. In an embodiment the tourniquet is simple and cheap to manufacture so that it can be disposable. However, this does not preclude the tourniquet from being reused, ideally after sterilisation.

The elongate member can be of any suitable size. For example, it may be desirable to have a smaller tourniquet for use on a child and a larger version for use on an adult. The tourniquet may also be used in veterinary applications.

The second end of the elongate member may be tapered for ease of passage through the aperture. This means it is easier for a person to use the tourniquet with one hand.

The invention may be performed in various ways and an embodiment will now be described with reference to the accompanying drawings in which:

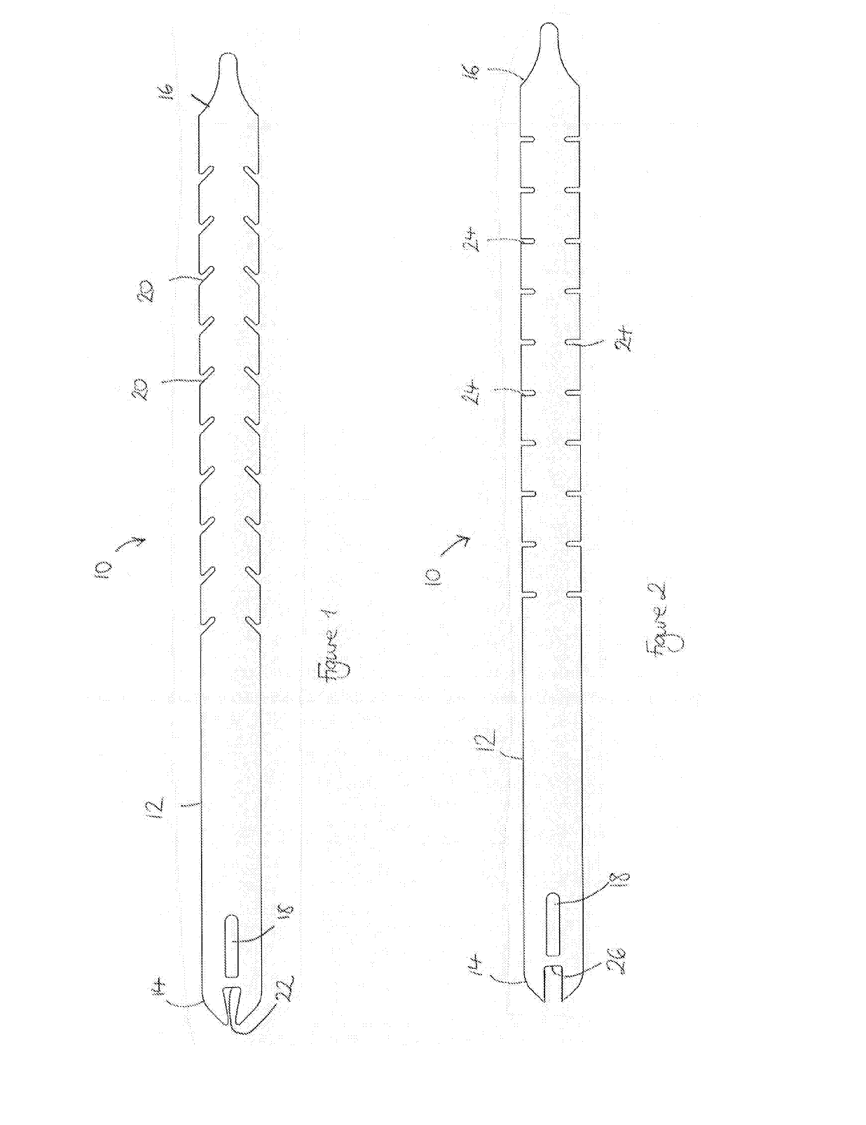

FIG. 1 shows a first embodiment of a tourniquet according to the present invention;

FIG. 2 shows a second embodiment of a tourniquet according to the present invention;

FIG. 3 shows a profile view of the tourniquet shown in FIG. 2;

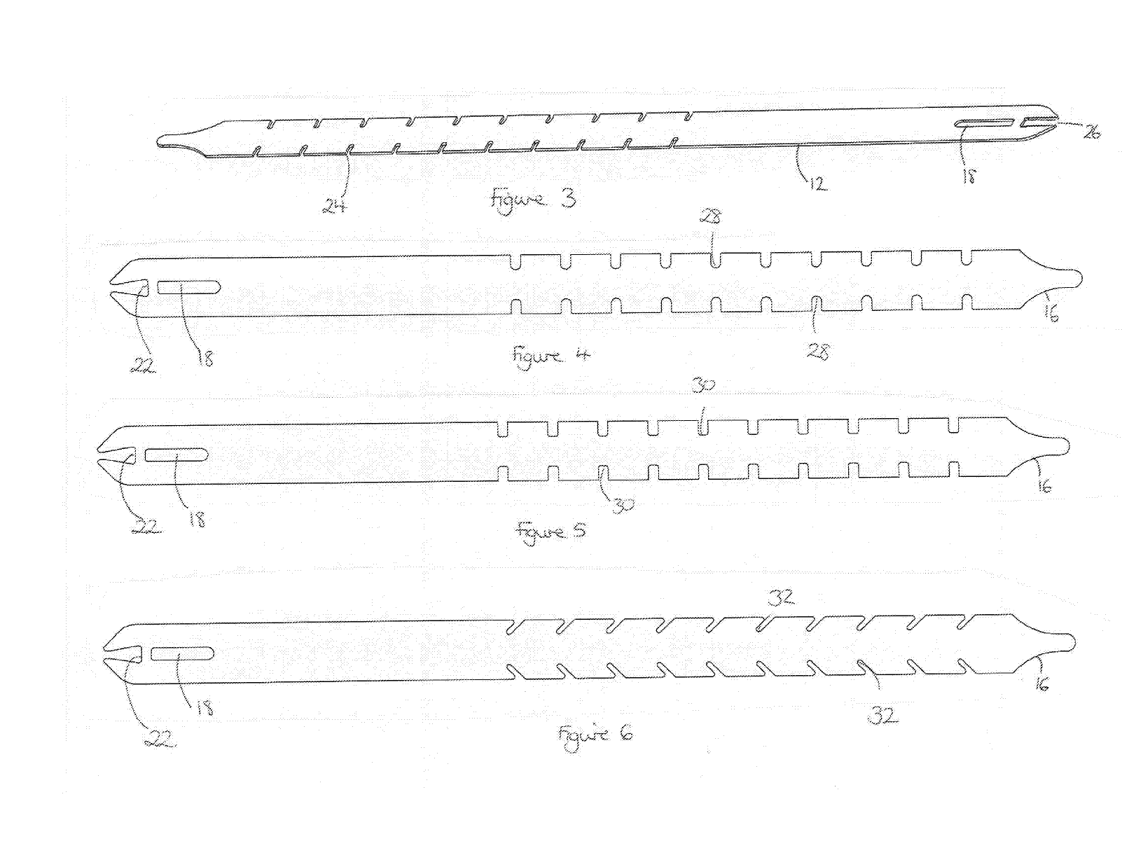

FIG. 4 shows a third embodiment of a tourniquet according to the present invention; FIG. 5 shows a fourth embodiment of a tourniquet according to the present invention; and FIG. 6 shows a fifth embodiment of a tourniquet according to the present invention.

FIG. 1 shows a tourniquet 10 having an elongate member 12 having a first end 14 and a second end 16. There is an aperture 18 proximate to the first end 14 through which the second end 16 is passed in order to wrap the tourniquet around a limb. The elongate member 12 has two substantially parallel edges 13, 15 having a plurality of engagement members 20 in the form of indentations which engage with the aperture 18 in use. In this embodiment, the engagement members 20 are equally spaced along both of the edges 13, 15 of the elongate member 12. The indentations 20 have a rounded inner surface to prevent tearing of the elongate member 12. The second end 16 is tapered for ease of passage through the aperture 18. The first end 14 defines a notch 22 which allows the first end 14 to deform, when the tourniquet is in use, which in turn deforms the aperture 18 to exert a gripping force on the engagement members 20.

FIG. 2 shows a tourniquet 10 having an elongate member 12 having a first end 14 and a second end 16. There is an aperture 18 proximate to the first end 14 through which the second end 16 is passed in order to wrap the tourniquet around a limb. The elongate member 12 has two substantially parallel edges 13, 15 having a plurality of engagement members 24 in the form of indentations which engage with the aperture 18. In this embodiment the engagement members 24 are opposite one another along the edges 13, 15 of the elongate member and are perpendicular to the length of the elongate member. The indentations 24 have a rounded inner surface to prevent tearing of the elongate member 12. The second end 16 is tapered for easy passage through the aperture 18. The first end 14 defines a notch 26 which allows the first end 14 to deform which in turn deforms the aperture 18 to exert a gripping force on the engagement members 24. FIG. 2 shows an alternative notch 26 which causes the first end 14 to deform which in turn causes the aperture 18 to deform. Which then exerts a gripping force on the engagement members 24.

FIG. 3 shows a perspective view of the tourniquet of FIG. 2. It can be seen that the tourniquet is relatively thin compared to the length which reduces the cost of production of the tourniquet but also means the tourniquet is lightweight and flexible thereby meaning it is easy to manipulate, particularly one handed.

FIGS. 4 to 6 show alternative embodiments of the tourniquet having different engagement members 28, 30 and 32.

The physician can pass the tapered second end 16 through the aperture 18 to form a loop. This loop can then be passed over the limb of a person. The physician pulls on the second end 16 so that the elongate member 12 is contacted with the end of the aperture 18 which is closest to the notch 22, 26. The physician pulls on the second end 16 or elongate body 12 that has passed through the aperture 18 until a sufficient tension is applied to the limb to render the vein turgid and one of the indentations 20, 24, 28, 30, 32 engages with the aperture 18. The physician can then release the tourniquet 10 from his grip so that he can use two hands to attend to the patient whilst the tourniquet remains in place. Once the physician has finished treating the patient he can quickly release the tourniquet by deforming the elongate member 12 so that it is dimensioned to pass through the aperture 18. Alternatively, he can pull on the first end 14 of the elongate member 12 proximate to the notch 22, 26 which causes the aperture 18 to deform the elongate member 12 so that it easily passes through the aperture 18. The tourniquet 10 can then be slid off the body part once it is sufficiently loosened or the second end 16 can be passed back through the aperture 18. This quick release minimises the length of time a patient is subjected to pressure around their body part.

The present invention overcomes the problems of the prior art by providing an improved tourniquet. The features described in the foregoing description, or the following claims, or the accompanying drawings may, separately or in any combination of such features, be utilised for realising the invention in diverse forms thereof.

1. A tourniquet of flexible material including:
an elongate member having a first and a second end,
said elongate member defining an aperture therethrough, proximate to the first end, for receiving the second end of the elongate member when in use,
said elongate member having at least one engagement member disposed along its length to engage the aperture wherein the first end defines a notch.

2. A tourniquet according to claim 1 wherein the at least one engagement member is configured to engage with the aperture defined by the first end of the elongate member.

3. A tourniquet according to claim 2 wherein the at least one engagement member is an indentation.

4. A tourniquet according to claim 3 wherein the elongate member has two substantially parallel edges along which the at least one indentation is located.

5. A tourniquet according to claim 1 wherein the elongate member includes a plurality of indentations.

6. A tourniquet according to claim 5 wherein the plurality of indentations are spaced along the elongate body on both of the substantially parallel edges so that there are two points of engagement with the aperture.

7. A tourniquet according to claim 1 wherein the flexible material is a silicone rubber.

8. A tourniquet according to claim 1 wherein the second end is tapered for ease of passage through the aperture.

9. (canceled)
